(19) United States
(12) Patent Application Publication
(10) Pub. No.: US 2012/0277528 A1
(43) Pub. Date: Nov. 1, 2012

(54) MOTHER-BABY ENDOSCOPE SYSTEM CONSISTING OF HARD MOTHER- HYSTEROSCOPE AND FLEXIBLE BABY-FALLOPOSCOPE

(76) Inventor: Tie Qiao, Guangzhou (CN)

(21) Appl. No.: 13/322,033

(22) PCT Filed: Jul. 15, 2010
(86) PCT No.: PCT/CN2010/075166

§ 371 Date: Nov. 22, 2011

(30) Foreign Application Priority Data
Jan. 21, 2010 (CN) 201010111183.6

(51) Int. Cl. A61B 1/005; A61B 1/04

(52) U.S. Cl. 600/109; 600/113

(57) ABSTRACT

The present invention relates to a medical apparatus, in particular, a mother-baby endoscope system consisting of hard mother-hysteroscope and flexible baby-falloposcope which comprises a hard mother-hysteroscope system and a flexible baby-falloposcope system. The hard mother-hysteroscope system comprises a hard hysteroscope and a sheathing canal of the hysteroscope. The flexible baby-falloposcope system is a flexible falloposcope which comprises a flexible front end and a liquid channel. At least one instrument channel is provided in the endoscope body of the hard hysteroscope such that the flexible front end of the flexible falloposcope can be place thereinto. The mother-baby endoscope system not only can be used to treat the lesions in uterine cavity, but also observe the lesions in oviduct, thereby it extends the using scope of hysteroscope in gynecological surgery and improves the accuracy of examination and treatment.

### FIELD OF TECHNOLOGY

The present invention relates to a medical apparatus, in particular, a mother-baby endoscope system consisting of hard mother-hysteroscope and flexible baby-falloposcope, which is a core medical apparatus to carry out gynecological endoscopic surgery in modern medicine.

### BACKGROUND

A hysteroscope is an optical apparatus for the observation, diagnosis and treatment of uterine cavity, in which all sorts of changes can be observed clearly such that an exact diagnosis can be made. The lesions in uterine cavity could be directly inspected using the hysteroscopic techniques, the pathological tissues could be localized, collected and examined, which makes the diagnosis accurate, prompt, overall and visualized. Thus the cancerous lesions may be early detected.

The hysteroscope is one of the main means to diagnose and treat the lesions in uterine cavity. However, the body of the hysteroscope is made of hard materials and has a large diameter, which will be unable to enter into the oviduct connected with uterine cervix, thus some lesions in the oviduct are difficult to be observed accurately.

### SUMMARY

It is an object of the present invention, for new tendency of gynecological diseases, to provide a mother-baby endoscope system combining the hard mother-hysteroscope with the flexible baby-falloposcope, which can simultaneously diagnose and treat the lesions in both uterine cavity and oviduct. The present invention can be used conveniently and reliably, and effectively improves the cure rate for uterine diseases.

With regard to the object, the present invention provides the following:

A mother-baby endoscope system consisting of hard mother-hysteroscope and flexible baby-falloposcope comprises a hard mother-hysteroscope system and a flexible baby-falloposcope system. The hard mother-hysteroscope system comprises a hard hysteroscope and a sheathing canal connected with the hysteroscope. The hard hysteroscope comprises an endoscope body, an instrument channel connected with the endoscope body, an input port of an ocular, an input port of cold light source, an endoscope front end arranged at the front of the endoscope body, wherein the endoscope front end comprises an apex part of the endoscope. The sheathing canal for the hysteroscope comprises a sheathing canal body, a water inlet channel and a water outlet channel connected with the sheathing canal body, and a sheathing canal front end arranged at the front of the sheathing canal body. The flexible baby-falloposcope system is a flexible falloposcope which comprises a flexible front end and a liquid channel. At least one instrument channel is provided in the endoscope body of the hard hysteroscope such that the flexible front end of the flexible falloposcope can be placed thereinto.

In the present invention, the diameter of the instrument channel is less than or equal to 3.5 mm (≦3.5 mm). The front end of the flexible falloposcope is made of flexible fiber materials, the diameter of which is less than or equal to 3.0 mm (≦3.0 mm) and the length of which is less than or equal to 1000 mm (≦1000 mm). The diameter of the liquid channel of the falloposcope is less than or equal to 1.5 mm (≦1.5 mm), the liquid channel can be either a channel for the liquid injection to oviduct, or a channel which an instrument goes through.

In the present invention, an optical system of the flexible falloposcope can be either an electronic optical system or a non-electronic optical system. When a non-electronic optical system was utilized, a cold light source connector and an input port of an ocular are arranged at the back end of the flexible falloposcope. Whereas an electronic optical system was utilized, an image data output interface is provided at the back end of the flexible falloposcope.

In the present invention, a non-electronic optical system is used in the hard hysteroscope.

To improve the image quality of the hard hysteroscope, a miniature ultrasonic probe set is also provided on the hard hysteroscope. The miniature ultrasonic probe set comprises a probe driver and a miniature probe being composed of a miniature probe front end and an apex part thereof, wherein the miniature probe front end can be stretched out from the hard hysteroscope through the instrument channel thereof, then the miniature ultrasonic probe set conducts ultrasound scan in real-time to get the images to each layer of uterine walls and organs surrounding the uterus.

Alternatively, the hard hysteroscope can use an optical system conducted by an electronic image processor. The optical system conducted by the electronic image processor is an electronic CCD optical system, disposed with CCD chip within ¼″ (<¼″), and at least 480,000 effective pixels, and lens with field angle 100° or above. CCD chip is arranged at the apex part of endoscope body.

Compared with the prior art, the benefits of the present invention are:

The mother-baby endoscope system according to the present invention is a combination of flexible falloposcope and hard hysteroscope, which not only can be used to treat the lesions in uterine cavity, but also observe the lesions in oviduct, thereby it extends the using scope of hysteroscope in gynecological surgery and improves the accuracy of examination and treatment.

### BRIEF DESCRIPTION OF THE DRAWINGS

FIG. 1 is a schematic diagram of a mother-baby endoscope system consisting of hard mother-hysteroscope and flexible baby-falloposcope according to the present invention.

FIG. 2 is a schematic diagram of the hard mother-hysteroscope according to the present invention.

FIG. 3 is a schematic diagram of the flexible baby-falloposcope according to the present invention.

FIG. 4 is a schematic diagram of a mother-baby endoscope system consisting of hard mother-hysteroscope and flexible baby-falloposcope in surgery according to the present invention.

### DETAILED DESCRIPTION

An embodiment of the present invention will be hereinafter described with reference to the drawings:

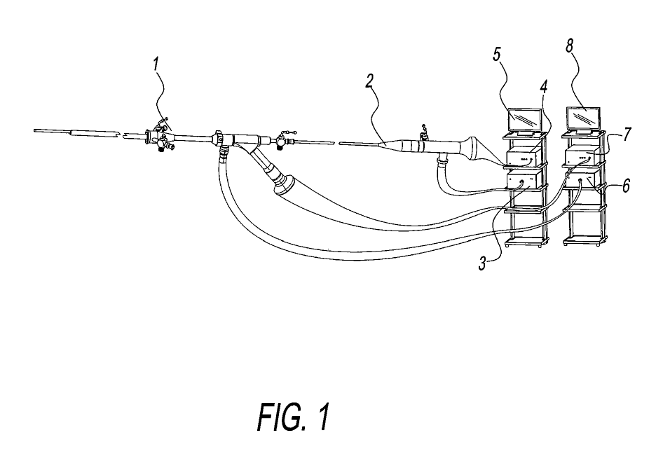

As shown in FIG. 1, the mother-baby endoscope system consisting of hard mother-hysteroscope and flexible baby-falloposcope described in the present invention comprises a hard mother-hysteroscope system and a flexible baby-falloposcope system, wherein the hard mother-hysteroscope system comprises a hard mother-hysteroscopte 1, a video camera system 7, a monitor 8 and a cold light source host machine 6. The flexible baby-falloposcope system comprises a flexible falloposcope 2, a video camera system 4, a monitor 5 and cold light source host machine 3.

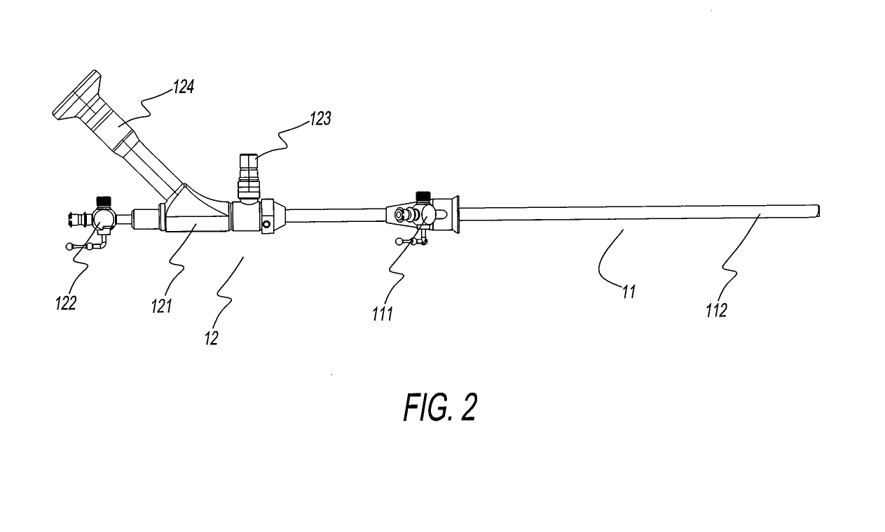

As shown in FIG. 2, the hard mother-hysteroscopte 1 described in the present invention comprises a hard hysteroscope 12 and a sheathing canal 11 connected with the hysteroscope, wherein the hard hysteroscope 12 comprises an endoscope body 121, an instrument channel 122 connected with the endoscope body 121, an input port 124 of the ocular, an input port 123 of cold light source, an endoscope front end arranged at the front of endoscope body and an apex part of the endoscope. The sheathing canal 11 for the hysteroscope comprises a sheathing canal body 111, a water inlet channel and a water outlet channel connected with the sheathing canal body 111, and a sheathing canal front end 112 arranged at the forefront of the sheathing canal body.

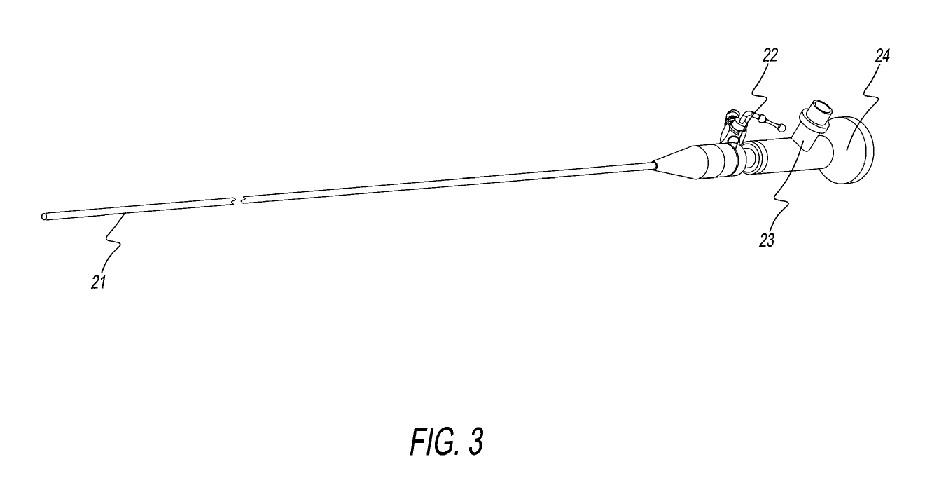

As shown in FIG. 3, the flexible baby-falloposcope 2 is a flexible falloposcope which comprises a flexible front end 21 and a liquid channel. In the present invention, at least one instrument channel is provided in the endoscope body 121 of the hard hysteroscope such that the flexible front end 21 of the flexible falloposcope can be placed thereinto. It can be seen by above description, the instrument channel 122 is the instrument channel for the access of the flexible front end 21 of flexible falloposcope, and the diameter of the channel is less than or equal to 3.5 mm (≦3.5mm). The diameter of front end of the flexible falloposcope is less than or equal to 3.0 mm (≦3.0 mm). The design is based upon anatomical evidence that the diameter of human oviduct is usually less than 3.0 mm (<3.0 mm), thus the diameter of the front end 21 of flexible falloposcope should be less than or equal to 3.0 mm (≦3.0 mm) and the length of the front end 21 is less than or equal to 1000 mm (≦1000 mm). A channel, having a diameter within 1.5 mm (≦1.5 mm), is provided in hysteroscope, which can be either a channel for the liquid injection to oviduct, or a channel which an instrument passes through.

In the present invention, the optical system of the flexible falloposcope can be either an electronic optical system or a non-electronic optical system. When a non-electronic optical system was utilized, a cold light source connector and an input port of an ocular are arranged at the back end of the flexible falloposcope. Furthermore, when an electronic optical system was utilized, an image data output interface is provided at the back end of the flexible falloposcope.

Furthermore, in the present invention, a miniature ultrasonic probe set is also provided on the hard hysteroscope 12, wherein the miniature ultrasonic probe set comprises a probe driver and a miniature probe being composed of a miniature probe front end and an apex part thereof The miniature probe front end can be stretched out from the hard hysteroscope through the instrument channel thereof Alternatively, the endoscope body of the hard hysteroscope can also use the optical system conducted by the electronic image processor. The optical system conducted by the electronic image processor is an electronic CCD optical system, disposed with CCD chip within ¼″ (≦¼″), and at least 480,000 effective pixels, and lens with field angle 100° or above. CCD chip is arranged at the apex part of endoscope body. The optical system can be chosen and used by a doctor according to the actual situation of patients.

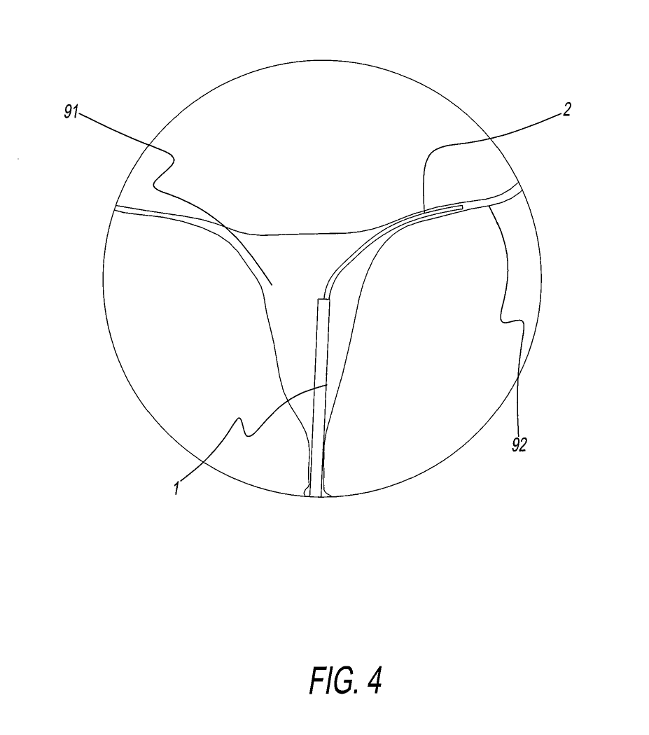

FIG. 4 is a working schematic diagram of the mother-baby endoscope system consisting of hard mother-hysteroscope and flexible baby-falloposcope. The hard mother—hysteroscopte 1 can conduct a general uterine surgery, diagnose and treat the lesions of the uterine cavity 91 after the hysteroscope 1 entered into the uterine cavity 91. After the hard mother—hysteroscopte 1 clearly found the entry of oviduct 92 in uterine cavity 91, the hysteroscopte 1 can be fixed by a holder, then the flexible falloposcope 2 enters into oviduct 92 from the instrument channel 122 of the hard mother-hysteroscopte 1. The water can subsequently be injected into the oviduct 92 through the channel 22 of the flexible falloposcope 2, thereby the oviduct 92 can clearly be observed, diagnosed and treated. The treatment device can also enter into oviduct 92 through the channel of the flexible falloposcope 2, and treats the lesions of oviduct 92 thereafter.

1. A mother-baby endoscope system consisting of hard mother-hysteroscope and flexible baby-falloposcope, wherein said system comprises a hard mother-hysteroscope system and a flexible baby-falloposcope system; Said hard mother-hysteroscope system comprises a hard hysteroscope and a sheathing canal connected with the hysteroscope, and the hard hysteroscope comprises an endoscope body, an instrument channel connected with the endoscope body, an input port of an ocular, an input port of cold light source, an endoscope front end arranged at the front of the endoscope body and an apex part of the endoscope, and the sheathing canal for the hysteroscope comprises a sheathing canal body, a water inlet channel and a water outlet channel connected with the sheathing canal body, and a sheathing canal front end arranged at the front of the sheathing canal body; The flexible baby-falloposcope system is a flexible falloposcope which comprises a flexible front end and a liquid channel, at least one instrument channel is provided in the endoscope body of the hard hysteroscope such that the flexible front end of the flexible falloposcope can be placed thereinto.

2. The mother-baby endoscope system of claim 1, wherein the diameter of the instrument channel is less than or equal to 3.5 mm.

3. The mother-baby endoscope system of claim 2, wherein the front end of the flexible falloposcope is made of flexible fiber materials, and the diameter of said front end is less than or equal to 3.0 mm, the length of the front end is less than or equal to 1000 mm, and the diameter of the liquid channel of the falloposcope is less than or equal to 1.5 mm.

4. The mother-baby endoscope system of claim 3, wherein an optical system of said flexible falloposcope can be either an electronic optical system or non-electronic optical system.

5. The mother-baby endoscope system of claim 4, wherein a cold light source connector and an input port of an ocular are arranged at the back end of the flexible falloposcope.

6. The mother-baby endoscope system of claim 4, wherein an image data output interface is provided at the back end of the flexible falloposcope.

7. The mother-baby endoscope system of claim 1, wherein a miniature ultrasonic probe set is provided on the hard hysteroscope, and the miniature ultrasonic probe set comprises a probe driver and a miniature probe being composed of a miniature probe front end and an apex part thereof; Said miniature probe front end can be stretched out from the hard hysteroscope through the instrument channel thereof.

8. The mother-baby endoscope system of claim 1, wherein the endoscope body of the hard hysteroscope utilizes an optical system conducted by an electronic image processor.

9. The electronic hard hysteroscope system with channel of claim 8, wherein the optical system conducted by the electronic image processor is an electronic CCD optical system, disposed with CCD chip within ¼″, and at least 480,000 effective pixels, and lens with field angle 100° or above; CCD chip is arranged at the apex part of endoscope body.
